(19) United States
(12) Patent Application Publication
(10) Pub. No.: US 2003/0082180 A1
(43) Pub. Date: May 1, 2003

(54) Combination of compounds that inhibit the biological effects of tnf-alpha and cd95l in a medicament

(76) Inventors: Peter Krammer, Heidelberg (DE); Ana Martin-Villalba, Heidelberg (DE)

Correspondence Address: INTELLECTUAL PROPERTY / TECHNOLOGY LAW, PO BOX 14329, RESEARCH TRIANGLE PARK, NC, 27709, US

(21) Appl. No.: 10/149,335

(22) PCT Filed: Dec. 6, 2000
(86) PCT No.: PCT/DE00/04371

(30) Foreign Application Priority Data
Dec. 7, 1999 (DE) 199 59 049.4

(51) Int. Cl. A61K 39/395

(52) U.S. Cl. 424/145.1; 424/141.1

(57) ABSTRACT

The invention relates to medicaments which contain one or more compounds inhibiting the biological effects of TNF-α and CD95L, e.g. by blocking the binding of these ligands to their natural receptors thus eliminating signal transduction. These compounds are preferably a neutralizing anti-TNF-α antibody and a neutralizing anti-CD95L antibody. The invention also relates to the use of the above compounds for preventing or treating an apoplectic stroke or heart attack.

The present invention relates to a medicament which contains one or more compounds inhibiting the biological effects of TNF-α and CD95L, e.g. by blocking the binding of these ligands to their receptors to thus eliminate signal transduction. These compounds are preferably a neutralizing anti-TNF-α antibody and a neutralizing anti-CD95L antibody. The present invention also relates to the use of the above compounds for preventing or treating an apoplectic stroke or heart attack.

Death caused by apoplectic stroke is the cause of death ranking third in most Western states. Moreover, the handicaps caused by apoplectic strokes, e.g. paralyses, virtually rank first among the monocausal handicaps. Up to now, treating an apoplectic stroke has predominantly consisted of a helping care and the prevention of further complications caused by this. The current experimental and clinical data indicate that the therapeutic window is variable and can also exceed 6 to 8 hours. This variable interval is determined by the ischemic penumbra. This is an area surrounding the center of the ischemic lesion, and hours may pass until this area necroses. Indications that the neurons in the ischemic penumbra suffer from apoptosis are now increasing in number. Thus, the therapies based on a neuroprotective strategy, i.e. aiming at a suppression of apoptosis, are most promising.

Two members of the tumor necrosis factor (TNF) family, CD95 (also referred to as Fas or ApoI) and the TNF receptor 1 (TNF-R1, also referred to as p55 or CD120a) are often involved in triggering apoptosis. These receptors distinguish themselves by a homologous cytoplasmic amino acid sequence (the “death domain”) which is of decisive significance for transducing the apoptotic signal. The natural ligands CD95L and TNF are structurally related type II transmembrane proteins. Binding the trimerized ligand to the receptor results in recruiting the FADD protein (“fass-associated death donmain”, also referred to as MORT1). Having recruited FADD, Caspase-8 is activated by self-cleavage and finally the cells go through apoptosis due to the activation of downstream effector caspases.

Following ischemia in the brain, the expression of TNF, CD95L and CD95 is increased in the ischemic penumbra. However, the role which TNF plays in connection with damage correlated with cerebral ischemia is disputed, in any case the administration of TNF prior to the ischemic infarct reduced the size of the infarct significantly. The role of CD95/CD95L appears to be rather clearly detrimental. However, it has been fully unclear by now whether these two ligand/receptor systems cooperate or work against each other when ischemia is induced. In any case, there is no satisfactory therapy thus far to prevent an ischemia in the brain or an apoplectic stroke in endangered persons or at least prevent, or at least reduce, the damage occurring in this connection.

Therefore, the present invention is based on the technical problem of providing a product by means of which—e.g. in endangered persons—ischemia in the brain or an apoplectic stroke can be, prevented and/or the damage occurring in this connection can be prevented or reduced.

This technical problem is solved by providing the embodiments characterized in the claims.

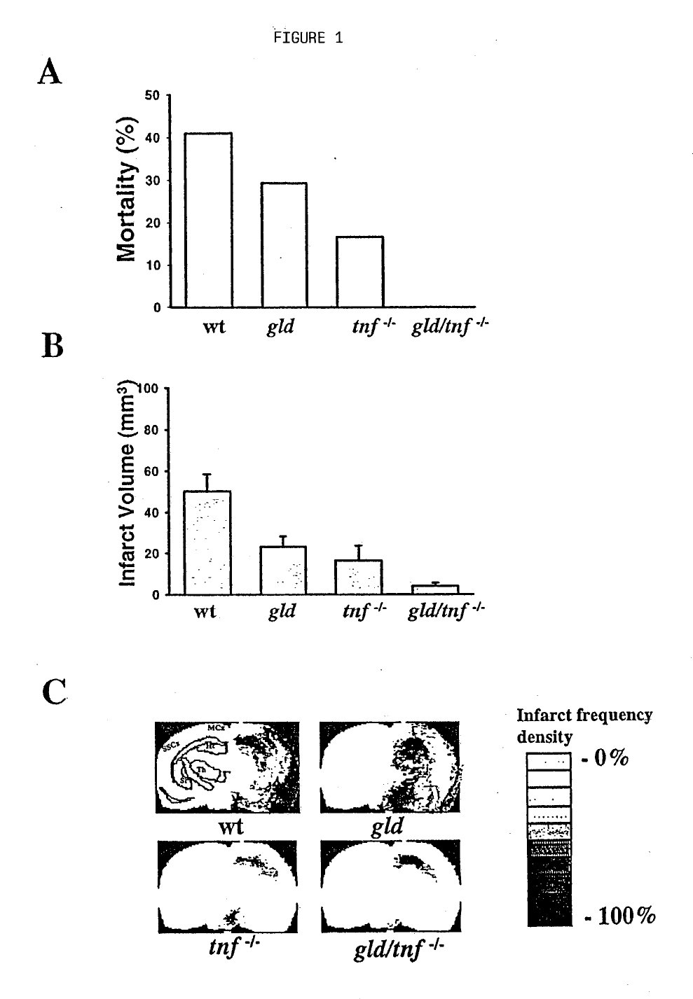

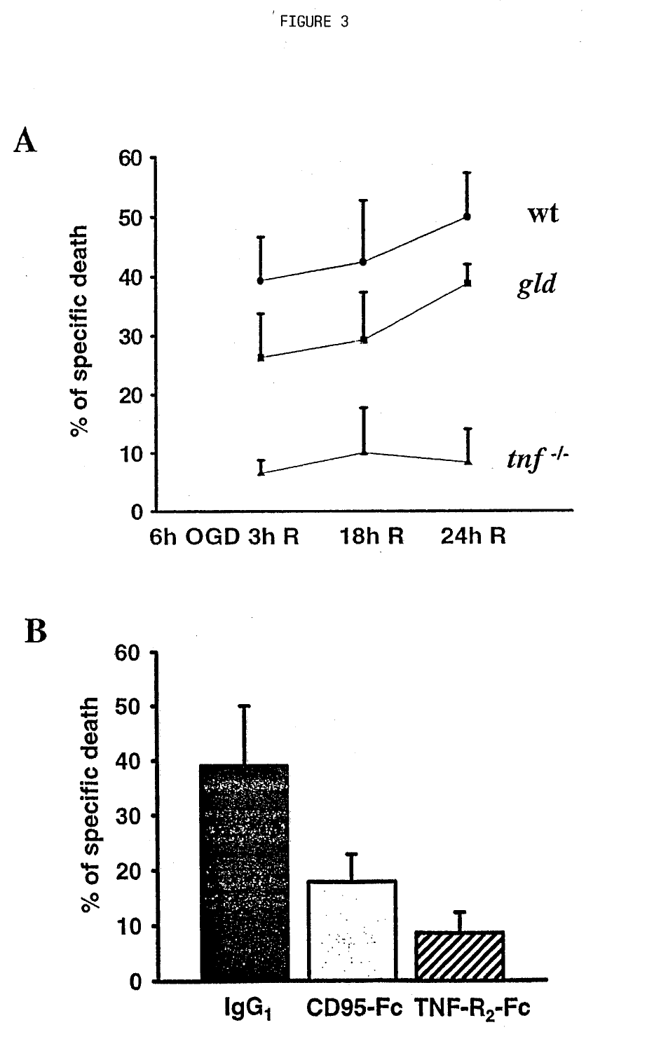

It was possible to show in the present invention that the two ligands CD95L and TNF trigger synergistically cell death after an ischemia. This role, promoting cell death, of CD95L and TNF is not based on modifications in the blood glucose content or hemodynamic parameters providing non-specific neuronal protection. The experiments show that CD95L and TNF play a role in both ischemic and inflammatory damage of the brain. Neurons deficient as to TNF or a functional CD95L (tnf^{-/-} neurons or gld neurons) showed partial protection from death caused by ischemia (FIG. 3a). This protection could also be shown in WT neurons in which TNF and CD95L had been withdrawn from the system by receptor proteins used as a “bait”. In vitro protection was greater in tnf^{-/-} neurons than in gld neurons. In the in vivo situation, however, the infarct volumes in tnf^{-/-} and gld mice showed no significant difference. Yet, in tnf^{-/-} mice less granulocytes were recruited to the ischemic area as compared to gld mice. The similar potency of CD95L as regards the promotion of ischemic damage can be explained by its, additional ability of activating the machinery of cytotoxic granulocytes—granulocytes convey direct cytolysis of CD95L+ cells. In any case, CD95L and TNF showed a synergistic effect in cerebral damage after an apoplectic stroke. Finally, it turned out that the neuronal protection shown by gld, tnf^{-/}⁻ and gld/tnf^{-/-} (made visible by means of the infarct volume; FIG. 1b) ran parallel to the death rate of these mice within the period of observation (FIG. 1a). Thus, the reduction of the cerebral damage increased the survival rate of the animals. The striking neuronal protection observed in gld/tnf^{-/-} mice shows that TNF-α/TNF receptor and CD95L/CD95 represent pharmacological objectives for the prevention/treatment of apoplectic stroke. Nowadays, strategies as regards neuronal protection aim at maintaining the vitality of the ischemic neurons until reperfusion can usually be re-established. However, cerebral reperfusion is followed by a destruction and another expansion of the zone of infarct. Hence it can be assumed that protection from both damage caused by reperfusion and ischemic cell death can be considered a model therapy of apoplectic stroke. On account of the results obtained in the present invention it can be assumed that this can be achieved by inhibiting or neutralizing the biological effect of TNF-α and CD95L.

Thus, an embodiment of the present invention relates to a medicament containing one or more compounds inhibiting the biological effects of TNF-α and CD95L.

The expression “compounds inhibiting the biological effects of TNF-α and CD95L” used herein relates to all the compounds which can fully or at least substantially inhibit or neutralize the biological effects of TNF-α and CD95L. For example, the effect may be based on suppressing the binding of TNF-α and CD95L to their natural receptors and therefore the thus caused signal transmissions. This can be achieved e.g. by using antibodies binding to either TNF-α per se or the TNF-α receptor or to CD95L per se or CD95 so as to block the binding of TNF-α and CD95L to the receptors. It is also possible to use small molecular substances which interfere with the binding of TNF-α to the TNF-α receptor or with the binding of CD95L to the CD95L receptor. Furthermore, analogous substances, e.g. of the extracellular, soluble part of the receptor or modified TNF-α or CD95L, e.g. a competitive or non-competitive antagonist, can also be used, the (preferred) binding of TNF-α or CD95L to the receptor analog or the (preferred) binding of the antagonist to the natural receptor serving for reducing or fully eliminating the binding of biologically active TNF-α or CD95L to the natural receptor.

In a preferred embodiment of the medicament according to the invention, the compounds preventing the binding of TNF-α and CD95L to their natural receptors, are a neutralizing anti-TNF-α antibody and a neutralizing anti-CD95L antibody or fragments thereof, preferably monoclonal antibodies or fragments thereof.

Methods of obtaining such antibodies are known to the person skilled in the art and comprise e.g. with respect to polyclonal antibodies the use of TNF-α or CD95L or a fragment thereof or a synthetic peptide derived from the amino acid sequence as an immunogen for immunizing suitable animals and obtaining serum. Methods of preparing monoclonal antibodies are also known to the person skilled in the art. For this purpose, e.g. cell hybrids are prepared from antibody-producing cells and bone marrow tumor cells and cloned. Thereafter, a clone is selected which produces an antibody specific to TNF-α or CD95L. This antibody is then prepared. Examples of cells which produce antibodies are spleen cells, lymph node cells, B lymphocytes, etc. Examples of animals which can be immunized for this purpose are mice, rats, horses, goats and rabbits. The myeloma cells can be obtained from mice, rats, humans or other sources. Cell fusion can be carried out by the generally known method by Köhler and Milstein, for example. The hybridomas obtained by cell fusion are screened by means of TNF-α or CD95L according to the enzyme-antibody method or according to a similar method. Clones are obtained with the boundary dilution method, for example. BALB/c mice are given the resulting clones by intraperitoneal implantation, the ascites of the mouse is removed after 10 to 14 days, and the monoclonal antibody is purified by known methods (e.g. ammonium sulfate fractionation, PEG fractionation, ion exchange chromatography, gel chromatography or affinity chromatography). In the medicament according to the invention, the obtained antibody can be used directly or a fragment thereof can be employed. In this connection, the term “fragment” refers to all the parts of the monoclonal antibody (e.g. Fab, Fv or “single chain Fv” fragments) which have an epitope specificity the same as that of the complete antibody.

In a particularly preferred embodiment, the monoclonal antibodies contained in the medicament according to the invention are antibodies derived from an animal (e.g. mouse), humanized antibodies or chimeric antibodies or fragments thereof. Chimeric antibodies resembling human antibodies or humanized antibodies have a reduced potential antigenicity, however, their affinity to the target is not lowered. The production of chimeric and humanized antibodies or of antibodies resembling human antibodies was described in detail (see e.g. Queen et al., Proc. Natl. Acad. Sci. USA 86 (1989), 10029, and Verhoeyan et al., Science 239 (1988), 1534). Humanized immunoglobulins have variable framework regions derived substantially from human immunoglobulin (referred to as acceptor immunoglobulin) and the complementarity of the determining regions derived substantially from a non-human immunoglobulin (e.g. of a mouse) (referred to as donor immunoglobulin). The constant region(s) originates), if present, also substantially from a human immunoglobulin. When administered to human patients, the humanized (and the human) anti-TNF-α antibodies or anti-CD95L antibodies offer a number of advantages over antibodies from mice or other species: (a) the human immune system should not regard the framework or the constant region of the humanized antibody as foreign and therefore the antibody response to such an injected antibody should be less than that to a completely foreign mouse antibody or a partially foreign chimeric antibody; (b) since the effector region of the humanized antibody is human, it might interact better with other parts of the human immune system, and (c) injected humanized antibodies have a half life which is substantially equivalent to that of naturally occurring human antibodies, which enables doses to be administered smaller and less frequent than those of antibodies from other species.

In another preferred embodiment the medicament according to the invention contains compounds which are (a) a TNF-α antagonist and a CD95L antagonist, (b) a soluble TNF-α receptor and a soluble CD95 receptor or parts thereof or (c) mixtures of one or both compounds from (a) and (b). The person skilled in the art knows how to provide such compounds. In particular, reference is made to Applicant's German patent P 44 12 177 and the study by Dhein et al., Nature 373 (1995), 438-441.

The compounds which neutralize the biological effect of TNF-α and CD95L, are optionally administered in combination with a pharmaceutically compatible carrier. Examples of suitable carriers are known to the person skilled in the art and they comprise e.g. phosphate-buffered salt solutions, water, emulsions, wetting agents, etc. The medicaments containing these compounds can be administered orally or preferably parenterally. The methods of parenteral administration comprise the topical, intra-arterial, intra-muscular, subcutaneous, intramedullary, intrathekal, intraventricular, intravenous, intraperitoneal or intranasal administration. A suitable dose is determined by the attending physician and depends on various factors, e.g. on the age, sex and weight of the patient, the stage and extent of the apoplectic stroke or heart attack, the kind of administration, etc.

Another embodiment of the present invention relates to the use of the above compound(s) of inhibiting or neutralizing the biological effect of TNF-α and the biological effect of CD95L to prevent or treat an apoplectic stroke or heart attack. As to the compounds suited for this use all of the above-described compounds can be employed, preferably a neutralizing anti-TNF-α antibody and a neutralizing anti-CD95L antibody or fragments thereof. Monoclonal antibodies or fragments thereof are particularly preferred, and antibodies derived from an animal, humanized antibodies or chimeric antibodies or fragments thereof are most preferred. In an alternative embodiment the compounds are (a) a TNF-α antagonist and a CD95L antagonist, (b) a soluble TNF-α receptor and a soluble CD95 receptor or parts thereof, or (c) mixtures of one or both compounds from (a) and (b). For use in the prevention or treatment of a heart attack it may be useful—above all as a function of the severity of the clinical picture—to combine the compounds; according to the invention also with thrombolytic preparations. Suitable thrombolytic preparations are known in the art and comprise e.g. acetylsalicylic acid, nitroglycerin, coumarin, heparin, heparinoids, fibrinolytic agents, etc.

By way of supplement, it is pointed out that a medicament according to the invention is not only suited to prevent ischemia in the brain or an apoplectic stroke or heart attack and/or prevent or reduce the resulting damage but can also be used generally in neurodegenerative diseases, such as Alzheimer's disease, Parkinson's disease and bone marrow trauma.

The figures show:

FIG. 1: Ischemic Cerebral Damage is Reduced in gld, tnf^{-/-} and gld/tnf^{-/-} Mice

(A) mortality (in %) within 24 hours after occlusion of the middle cerebral artery (MCA) in wild-type (n=14), gld (n=17), tnf^{-/- (n=}13) and gld/tnf^{-/-} (n=15) mice, (B) Infarct volume after transient focal ischemia in wild-type (n=9), gld (n=8), tnf^{-/-} (n=7) and gld/tnf^{-/-} (n=8) mice. The animals underwent MCA occlusion for 90 minutes and reperfusion for 24 hours as described above. Coronal cryostat sections which had a thickness of 20 μm and were 400 μm apart in each case were silver-stained. The infarct volume was determined by the numerical integration of areas having marked paleness with respect to the thickness of the sections. The data are shown as mean value±S.E.M. The significance was determined by comparison of gld, tnf^{-/-} and gld/tnf^{-/-} mice with wild-type mice using the “Mann Whitney's” test (P<0.01, P001 or P<0.001). (C) The picture analysis of the regional infarct frequencies of the coronary area with bregma −2.3 mm of wild-type, gld, tnf^{-/-} and gld/tnf^{-/-} mice clearly shows a relative unaffected area of the motorial and somatosensory cortex and the striatum in gld mice and the entire adjacent neocortex, striatum and thalamus in tnf^{-/-} mice. In gld/tnf^{-/-} mice the hippocampus was virtually the only affected area. MCx: motorial cortex; SSCx: somatosensory cortex; Hc: hippocampus; Th: thalamus; St: striatum.

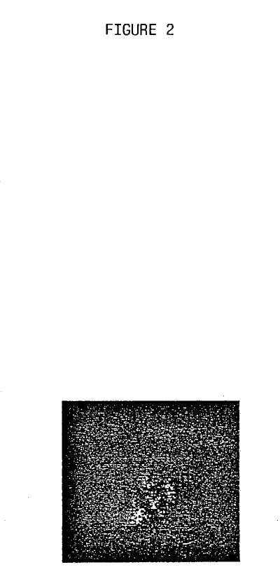

FIG. 2: CD95L and TNF are Localized in the Ischemic Penumbra Within the Same Cell

Double immunofluorescence analysis was carried out with cerebral sections from wild-type mice which had undergone 90-minute MCA occlusion and 24-hour reperfusion. Cells in the ischemic preumbra were positive as regards CD95L and TNF (FITC: green; Cy3red; both together: yellow)

FIG. 3: Protection from Death by Ischemia in the in vitro Model for a Vascular Apoplectic Stroke

(A) Cortical neurons of wild-type, gld, tnf^{-/-} and gld/tnf^{-/-} mice underwent 6-hour oxygen/glucose deprivation (OGD) and increasing reperfusion periods (R). The specific death was determined after 3, 18 and 24 hours of reperfusion (R). (B) cortical neurons of wild-type animals were incubated with CD95-Fc proteins and TNF-R2-Fc proteins (20 μg each) prior to induction of 6-hour OGD and 18-hour reperfusion. The specific death was determined at the end of the reperfusion period. Incubation with control immunoglobulin (IgF₁) had no influence as regards the neurotoxicity of the cultures. The cell death was estimated by means of the “trypan blue exclusion” method and is given in each case as average value±standard deviation (n=3). The specific cell death was calculated as described in the below examples.

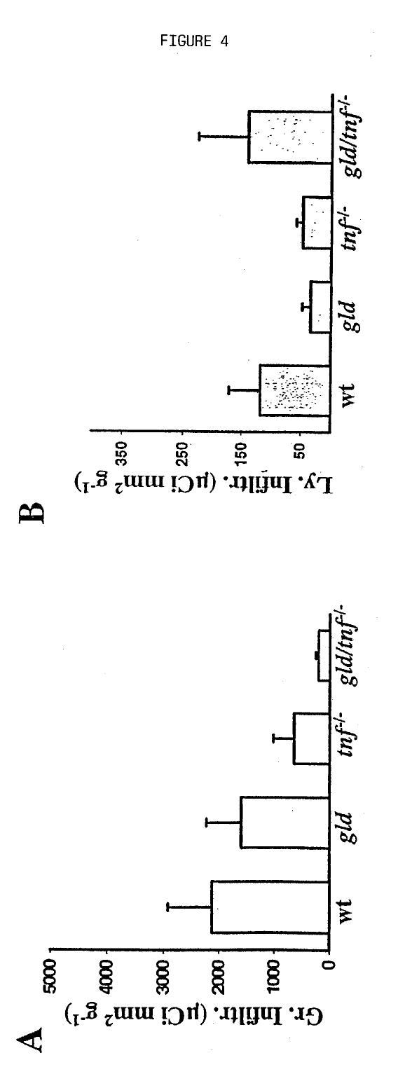

FIG. 4: Infiltration of the Ischemic Brain with Cells Involved in Inflammatory Processes

Infiltration with granulocytes (Gr.Infilt.; A) or lymphocytes (Ly.Infiltr.; B) of the ischemic brain of wild-type, gld, tnf^{-/-} and gld/tnf^{-/-} mice (n=3 each) was quantified by means of autoimmunoradiography. Cerebral sections from brain subjected to focus-like ischemia (90-minute MCA occlusion and 25-hour reperfusion) were incubated with primary antisera against GR1 (for granulocytes) and CD3 (for lymphocytes). The staining was determined by means of autoradiography. Inflammatory infiltrates in the ischemic hemisphere were quantified by measuring the area and the optical density (OD) of the infiltrates. The results are shown as average values±S.E.M. (n=3).

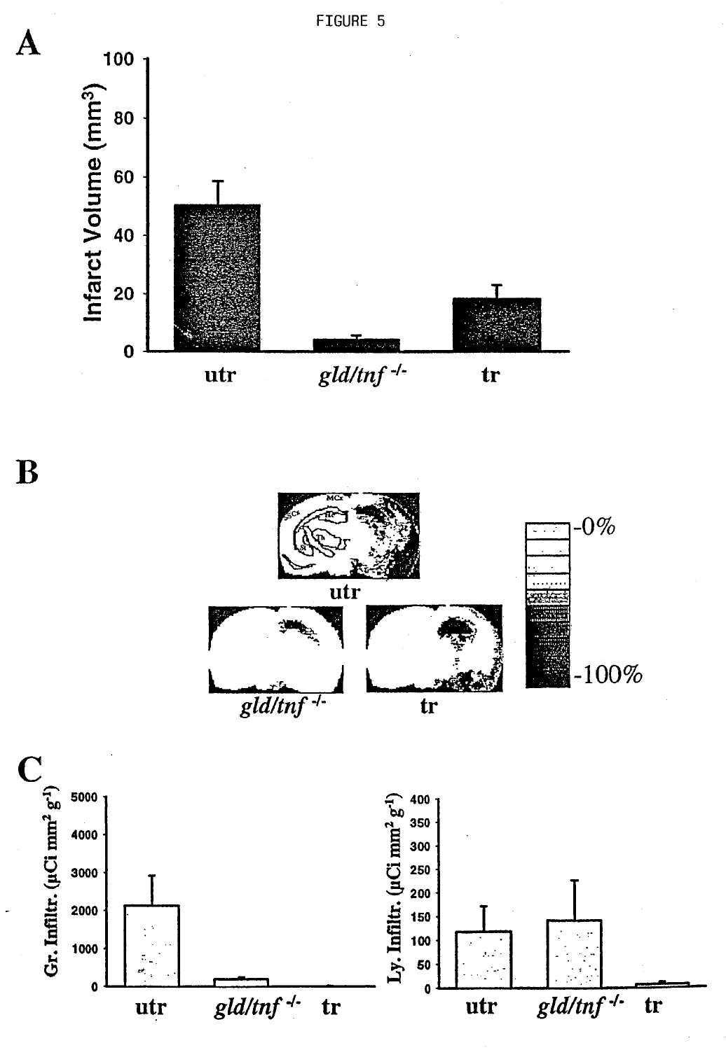

FIG. 5: Infarct Expansion and Inflammatory Infiltrates of the Ischemic Brain are Significantly Reduced by anti-TNF and anti-CD95L Antibodies

Untreated animals (n=9; utr) or animals treated with antibodies against TNF and CD95L (n=8; tr) and gld/tnf^{-/-} animals (n=8) underwent 90-minute MCA occlusion and 24-hour reperfusion. Coronary cryostat sections (thickness: 20 μm; distance from one another 400 μm each) were silver-stained. The infarct volume was determined by numerical integration of the areas having marked paleness with the section thickness. The data are shown as average values±S.E.M. The significance was determined by comparison of the “tr” and gld/tnf^{-/-} mice with “utr” mice using the “Mann Whitney's” test (P<0.01 or P<0.0001). (B) Picture analysis of the regional infarct frequencies of the coronary section with bregma −2.3 mm from “utr”, “tr” and gld/tnf^{-/-} mice yielded a relative unaffected area of the motorial and somatosensory cortex and the striatum in “tr” mice. In the gld/tnf^{-/-} mice, the hippocampus was virtually the only affected area. MCx: motorial cortex; SSCx, somatosensory cortex; Hc: hippocampus; Th: thalamus; St: striatum; C; number of granulocytes (Gr.Infiltr.) and lymphocytes (Ly.Infiltr.) in the ischemic hemisphere were determined by means of autoimmunoradiography. Cerebral sections from animals exposed to focus-like ischemia (90-minute MCA occlusion and 24-hour reperfusion) were incubated with primary antisera against GR1 (for granulocytes) and CD3 (foy lymphocytes). The stainings were made visible by means of autoradiography. Inflammatory infiltrates in the ischemic hemisphere were quantified by measuring the area and the optical density (OD) of the infiltrates. The data are shown as average values±S.E.M. (n=3).

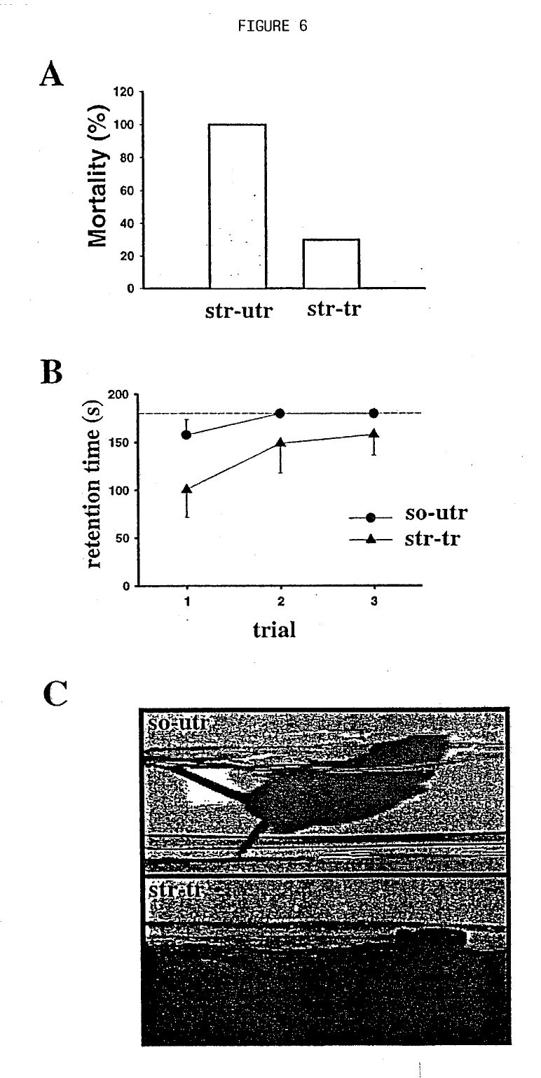

FIG. 6. Neutralization of CD95L and TNF Reduces the Mortaility and Improves the Motorial Performances of the Animals After an Apoplectic Stroke

(A) Mortality (in %) within three days following MCA occlusion in untreated animals (n=5; str-utr) or animals treated with anti-TNF and anti-CD95L antibodies (n=10; str-utr). (B) Periods during which untreated pseudo-operated (so-utr; n=6) and treated apoplectic stroke animals (str-tr; n=5) could balance on a rod having a gradually increasing rotational speed (C) Initial experiences of “so-utr” and “str-tr” mice as regards swimming.

The below examples explain the invention.

### EXAMPLE 1

### General Method

(A) Ischemic animal model: In wild-type (n=14), gld (n=17), tnf^{-/-} (n=13) and gld/tnf^{-/-} (n=15) mice, all matched as regards age (average value: 100 days) and weight (average value: 24 g), a focus-like cerebral ischemia was induced by MCA occlusion as described by Zea Longa et at., Stroke 20 (1989), 84. A surgical nylon thread was wound from the lumen of the common carotid to the frontal cerebral artery to block the origin for MCA for 90 minutes. By removing the nylon thread the blood flow was established again. Sound anesthesia was carried out by means of ketamine/ROMPOM (150 mg/kg body weight each). The animals were kept anesthetized and the rectal temperature was kept at or around 37° C. by means of heat-generating lamps both during the Operations and the period of MCA occlusion. After various reperfusion periods, the animals underwent sound anesthesia again and were killed by cutting off their heads. In order to obtain physiological parameters, a cannula was introduced into the artery of the right thigh by constant anesthesia, the blood pressure was recorded continuously and samples as to the blood-gas content and glucose were withdrawn 15 minutes before, 1 hour after the commencement and 30 minutes after the termination of the MCA occlusion. The sex of the mice varied depending on the availability. In any case, the outcome of the infarct was not influenced by the sex of the animals. All of the mice had a C57BL/6 background to avoid the known differences as regards the susceptibility to an infarct as a function of the respective mouse strain (Connolly et al., Neurosurgery 38 (1996), 523). For the treatment experiments, 50 μg anti-CD95L antibodies MFL3 and anti-TNF antibodies V1q each were given by i.v. or i.p. injection 15 min. and 24 hours after the MCA occlusion.

(B) Measurement of infarct expansion: The mice were exposed to MCA filament occlusion for 90 min. and, as described above, reperfusion was carried out for 24 hours. The forebrains were cut and coronary cryostat sections (thickness: 20 μm, 400 μm apart in each case) were silver-stained. Briefly summarized, the following steps were taken: sections were impregnated with a silver nitrate/lithiumcarbonate solution for 2 min. and developed with a hydroquinone/formaldehyde solution for 3 min. Stained sections were scanned directly (MCID-M4, 3.0). The infarct volume was determined by numerical integration of the scanned area with marked paleness (corrected for cerebral edema×thickness of section using digital planimetry (Swanson et al., J. Cereb. Blood Flow Metab. 10 (1990), 290)). All of the data are given as average values±S.E.M. (average standard deviation). The significance was determined by means of the MANN-WITHNEY's t-test. In order to produce infarct frequency maps, the corresponding sections were scanned, infarcts were shown and projected onto a mask. The average was determined by means of a “Scion Image β 3.b”.

(C) Immunohistochemical Analysis of CD95L and TNF Expression

Coronary cryostat sections (20 μm) from wild-type mice which had undergone 90-minute MCA occlusion and 24-hour reperfusion were prepared for immunohistochemical analysis. The sections were incubated with a monoclonal antibody against CD95L (P62) or a monoclonal antibody against TNF (Hartung) Immunoresponses with CD95L and TNF were made visible by either a secondary polyclonal FITC-labeled antibody or a secondary monoclonal Cy3-labeled antibody.

(D) Detection of infiltration of cells involved in inflammatory processes: Coronary cryostat sections (20 μm) from wild-type, gld, tnf^{-/-} and gld/tnf^{-/-} mice which had undergone 90-minute MCA occlusion and 24-hour reperfusion were prepared for immunoautoradiography. The sections were incubated with the monoclonal antibody against GR1 (Ly-1) or a monoclonal antibody against CD3 (Chemicon) for 24 hours. Thereafter, the sections were incubated with a ¹²⁵I-labeled secondary antibody. The radioautogram was prepared (together with a [¹²⁵I] standard set) on a Kodak “MinR1” X-ray film (exposition: 21 days). Granulcyte or lymphocyte infiltration in the ischemic hemisphere was determined by measuring the optical density and the area of the infiltrates with an image analysis system (MCID).

(E) Cell culture and experimental in vitro treatment: Primary neuronal cultures were prepared from fetal mice (from days 15 to 17). Briefly summarized, the following steps were taken: Cortical neurons were obtained following pulverization in the MEM medium with 20% horse serum, 25 mM glucose and 2 mM L-glutamine. This was followed by 30-minute cleavage in 0.025% trypsin/common salt solution. The cells were placed into plates having 24 wells coated with polyornithine. After 4 days, the cells were treated for another four days using citosine (5 μM) arabinoside to inhibit the proliferation of non-neuronal cells. Thereafter, the cell cultures were kept in MEM, 10% horse serum 5% fetal bovine serum, 25 mM glucose and 2 mM L-glutamine at 37° C. in a moistened CO₂ incubator (8%). The neurons were allowed to mature in culture for at least 8 days prior to their experimental use. The gliacyte portion in the cultures was below 10% (estimated by means of an antibody against “glial-fibrillary-acidic protein” (GFAP)).

(F) Oxygen/glucose deprivation in vitro: Combined oxygen/glucose deprivation was carried out. The culture medium was exchanged for MEM, 1% horse serum and 2 mM L-glutamine. The cultures were kept at 37° C. and 100% humidity for 6 hours in an anaerobic chamber containing the gas mixture 5% H₂/85% N₂/5% CO₂. The combined oxygen/glucose deprivation was terminated by removing the cultures from the chamber and adding horse serum, fetal bovine serum and glucose up to a final concentration of 10%, 5% and 25 mM, respectively. The cultures were then incubated at 37° C. for another 3, 18 or 24 hours in a moistened incubator which contained 8% CO₂ and atmospheric oxygen. Human IgG₁, CD95-Fc and TNF-R2-Fc (20 μM each) were added to the culture medium 5 min. prior to the induction of OGD. The percentage of cell death was determined by means of the “trypan blue deprivation” method and indicated as % specific cell death. It was calculated as follows:

$$\% specific cell death = \frac{( determined - spontaneous cell death )}{( 100 - spontaneous cell death )} \times 100$$

Spontaneous cell death was 12%±0.9 for neurons from the tnf-α knockout mice, 10%±0.73 for neurons from gld mice and 15%±0.87 for neurons from C57BL/6 wild-type mice. All of the data are given as average values±standard deviation (n=3).

(F) Motorial coordination: 12 to 16 week-old male C57BL/6 mice were placed on a fixed horizontal rod made of wood or Plexiglas and the time during which the animal balanced on the rod was measured. For the experiments with the rotary rod, the mouse was placed on a plastic roll coated with fine chippings and accelerated within 5 min. from 4 to 40 rpm. The balancing periods were recorded within a period of up to 180 seconds.

TABLE 1
Physiological variable before, during and after MCA occlusion in
wild-type (C57BL16), gld, tnf^{-/-}and gld/tnf^{-/-}mice.

| | Animal | Before | during | after |
|---|---|---|---|---|
| MABP | Wild-type | 95 ± 8 | 93 ± 10 | 95 ± 12 |
| (mmHG) | gld | 92 ± 12 | 95 ± 8 | 958 |
| | tnf^{-/-} | 77.5 ± 6 | 72 ± 9 | 1055 |
| | gld/tnf^{-/-} | 75 ± 4 | 82 ± 13 | 84 ± 12 |
| Arterial pH | wild-type | 7.22 ± 0.05 | 7.17 ± 0.04 | 7.2 ± 0.02 |
| | gld | 7.3 ± 0.07 | 7.22 ± 0.09 | 7.23 ± 0.02 |
| | tnf^{-/-} | 7.25 ± 0.04 | 7.26 ± 0.06 | 7.21 ± 0.01 |
| | gld/tnf^{-/31} | 7.16 ± 0.07 | 7.14 ± 0.11 | 7.13 ± 0.08 |
| Arterial | wild-type | 53.9 ± 5 | 58.4 ± 4 | 44.5 ± 3 |
| PaCO₂ | gid | 48.7 ± 5 | 51.4 ± 11 | 49.9 ± 1 |
| (torr) | tnf^{-/-} | 54.4 ± 1 | 47.2 ± 1 | 53.6 ± 1 |
| | gld/tnf^{-/-} | 64.3 ± 7 | 59.5 ± 7 | 57.8 ± 9 |
| Arterial PaO₂ | wild-type | 152 ± 26 | 82 ± 6 | 124 ± 11 |
| (torr) | gld | 158 ± 21 | 96 ± 18 | 86 ± 6 |
| | tnf^{-/-} | 118 ± 14 | 126 ± 14 | 98 ± 6 |
| | gld/tnf^{-/-} | 103 ± 8 | 84 ± 10 | 115 ± 6 |
| Hematocrit | wild-type | 35 ± 5 | 45 ± 1 | 39 ± 4 |
| | gld | 42 ± 4 | 40 ± 2 | 36 ± 4 |
| | tnf^{-/-} | 41 ± 1 | 46 ± 2 | 52 ± 1 |
| | gld/tnf^{-/-} | 40 ± 6 | 39 ± 8 | 35 ± 8 |
| Blood | wild-type | 206 ± 17 | 225 ± 17 | 217 ± 27 |
| glucose | gld | 153 ± 23 | 170 ± 21 | 133 ± 21 |
| (mg/dl) | tnf^{-/-} | 182 ± 16 | 242 ± 44 | 231 ± 25 |
| | gld/tnf^{-/31} | 216 ± 50 | 171 ± 43 | 187 ± 55 |

MABP: mean arterial blood pressure; blood gases (pH, PaCO₂ and PaO₂) and blood glucose were measured 15 mm. before, 1 hour after the beginning and 30 min. after the termination of the MCA occlusion. The data are given as average values ± standard deviation (all groups, n = 4). The significance was measured by comparing the gld, tnf^{-/-}and gld/tnf^{-/-}mice with wild-type mice by means of the student t-testl (P < 0.05).

### EXAMPLE 2

### CD95L and TNF Promote Synergistically Cell Death After Cerebral Ischemia

Mice carrying a well-calculated disruption of the tnf gene tnf^{-/-}, mice with mutated CD95L with the blocked ability of successful interaction with CD95 (gld), mice with a mutated CD95L and deficient for TNF (gld/tnf^{-/-}), and wild-type mice (WT), all of which had a C57BL/6 background, were used to study the role of TNF and CD95L and their possible interaction in the damage caused by ischemia in the brain. tnf^{-/-} and gld mice showed no development anomalies and only specific defects as regards immune responses. gld/tnf^{-/-} mice showed no structural or morphological anomalies and the cerebral anatomy determined by means of “Nissl” staining of the coronal cryostat sections appeared normal. WT, gld, tnf^{-/-} and gld/tnf^{-/-} mice underwent 90-minute MCA occlusion, and the glucose content measured before, during and after the CMA occlusion did not vary significantly between the individual animal groups (see Table 1) The mean infarct volume shown by wild-type animals was well in conformity with the one obtained with other groups in similar experiments. Data of mice showing no ischemic lesion or of mice which died before the observation period was over (41% wild-type, 29% gld and 17% tnf^{-/-} mice; FIG. 1a) were not added to analyses of the infarct volume.

In gld and tnf^{-/-} mice, the infarct volume was reduced significantly by about 54% and 67% as compared to wild-type mice (23.23±4.97 mm³, n=8 and 16.44±17.24 mm³, n=7 as compared to 50.11±8.38 mm³, n=9; for both P<0.01; FIG. 1b). Surprisingly, neuronal protection was strongly improved when both CD95L and TNF lacked. gld/tnf^{-/-} mice showed an average infarct volume significantly less than that of wild-type mice (3.97±1.52 mm³, n=8 and 50.11±8.38 mm³, n=9; P<0.0001; FIG. 1b).

The regional infarct distribution in the coronal plane was analyzed using the average determination of the infarct areas (coronal section at bregma −2.3 mm). The resulting frequency density map shows a relative unaffected area of the motorial and somatosensory cortex and the striatum in gld mice and a relative unaffected area of the entire contiguous neocortex, striatum and thalamus in tnf^{-/-} mice (FIG. 1c). In gld/tnf^{-/-} mice, the striatum, cortex and thalamus were not affected by the ischemic insult and the damage was mainly limited to the hippocampus (FIG. 1c).

CD95L and TNF are expressed in neurons of the ischemic penumbra. In order to study whether they are localized on the same cell, the expression of CD95L and TNF was analyzed by means of double immunofluorescence in cerebral sections from animals which had undergone 90-minute MCA occlusion and 24-hour reperfusion. In the ischemic penumbra, TNF was localized on CD95L-expressing cells (FIG. 2). Thus, TNF and CD95L could mutually increase their expression.

### EXAMPLE 3

### CD95L and TNF Support Neuronal Death and Inflammations

In order to be able to study in more detail the mechanism underlying ischemic cerebal damage by CD95L and TNF, the in vitro model of oxygen/glucose deprivation (OGD) was used. OGD in primary neuronal cultures is a common in vitro model to study early mechanisms in the case of damage caused by a vascular apoplectic stroke in a system which consists exclusively of neurons. Primary cortical neurons obtained from wild-type, gld, tnf^{-/-} and gld/tnf^{-/-} mice were subjected to 6-hour OGD and reperfusion for 3, 18 or 24 hours. As compared to wild-type neurons tnf^{-/-} neurons were substantially resistant to OGD/reperfusion-induced damage. Only about 10% of the tnf^{-/-} neurons died after OGD/reperfusion while up to 47% of the WT neurons died (FIG. 3a). At each studied point of time, the extent of specific cell death of the gld neurons was about 20% less than that of WT neurons (FIG. 3a). Neuronal cells derived from gld/tnf^{-/-} mice could not be cultured, which was presumably due to the specific in vitro situation.

tnf^{-/-} and gld mice showed normal development and anatomy of the brain. This obviously normal “cerebral phenotype” could be based on the presence of a compensatory mechanism which provides non-specific protection from cerebral ischemia. In order to rule out this possibility, CD95-FC and TNF-R2-Fc proteins were used for the treatment 15 min. prior to the induction of the OGD WT neurons. After 6-hour OGD and 18-hour reperfusion, the deprivation of either CD95L or TNF-α reduced the neurotoxicity of the cultures by 25% and 39%, respectively, as compared to controls treated with IgG₁ (immunoglobluin G₁) (FIG. 3b), Thus, the inhibition of the activity of TNF and CD95L can block specifically the OGD/reperfusion-induced neuronal death.

In the in vivo situation, cytokine production and molecular adhesive events occur shortly after the reduction of the cerebral blood flow. The TNF produced by the ischemic parenchyma is involved in the endothelial cell expression of cellular adhesive molecules, e.g. the intercellular “adhesion molecule-1” (ICAM-1), the vascular “cell adhesion molecule-1” (VCAM-1) and the “endothelial-leukocyte adhesion molecule-l” (E-selectin). Adhesion molecules facilitate the recruitment of cells involved in inflammatory processes to the ischemic lesion. Chemotactial properties which are similar in tumors were described for CD95L. According to these findings, the question arose whether in addition to the cell death-supporting role of CD95L and TNF following an ischemia these two molecules are also involved in the ischemic damage due to the recruitment of cells involved in inflammatory processes. In order to answer this question, the presence of infiltrating granulocytes and infiltrating lymphocytes were analyzed by means of autoimmunoradiography in the cerebral sections of wild-type, gld, tnf^{-/-}, and gld/tnf^{-/-} mice (n=3 each) which were subjected to focal ischemia (90-minute MCA occlusion and 24-hour reperfusion). Control stains without the first antibody or from cerebral sections of pseudo-operated animals were negative. The quantitative analysis of autoradiograms yielded a decreasing degree of granulocyte infiltration in gld, tnf^{-/-} and gld/tnf^{-/-} mice (arranged from the highest to the lowest infiltration degree), which was in conformity with the degree of cerebral damage (FIG. 4). The degree of lymphocyte infiltration was comparable in WT and gld/tnf^{-/-} mice and somewhat increased as compared to gld and tnf^{-/-} mice (FIG. 4). These data indicate a synergistic chemotactical effect of TNF and CD95L towards granulocytes following ischemia.

### EXAMPLE 4

### The Treatment with anti-TNF and anti-CD95L Antibodies Reduces Ischemic Damage

In order to put the findings from the genetically modified animals into practice, TNF and CD95L were neutralized in vivo following focal ischemia. In WT mice, an injection of anti-TNF and anti-CD95L antibodies induced i.v. (50 μg each) an about 60% reduction of the infarct volume as compared to untreated animals (18.04±4.87 mm³, n=8, in treated mice versus 50.11±8.38 mm³, n=9, P<001; FIG. 5a) 15 minutes after MCA occlusion. The regional infarct distribution in the coronal plane was determined by specifying the average of the infarct zones (coronal section at bregma −2.3 mm). The resulting frequency density map shows an almost exclusive involvement of the hippocampus and the piriform cortex in the ischemic lesion of treated animals (“stroke-tr”; FIG. 5b). Inflammatory infiltrates could hardly be detected in treated animals (FIG. 5c).

The functionality of the rescued neurons was studied by tests as regards the motorial coordination of treated mice after three days of reperfusion. For this purpose, anti-TNF and anti-CD95L antibodies (50 μg each) were given by i.p. injection 15 min and 34 hours after MCA occlusion. All of the untreated animals exposed to focal ischemia died before this period was over (“stroke-ur”; n=5) whereas the mortality in the treated group (“stroke-tr”; n=10) was only 30% (FIG. 6a). Six pseudo-operated untreated animals (so-utr”) and five treated mice which had undergone 90-minute occlusion and 3-day reperfusion were tested on a rotary rod and on stationary rods. The balancing capacity shown by the animals treated ischemically on the rotary rod did not differ significantly from that of the pseudo-operated animals (FIGS. 6b/c). The motorial coordination on stationary rods ran parallel to these results. Maintaining the balance concerning the axis was comparable in “so-utr” and “stroke-utr” animals. Even animals having a reduced balancing period on the rotary rod showed unimpaired balance concerning the axis when they were placed in water for the first time.

1. A medicament containing one or more compounds which inhibit the biological effect of TNF-α and CD95L, wherein the compounds are
a neutralizing anti-TNF-α antibody and a neutralizing anti-CD95L antibody or fragments thereof, or
(a) a TNF-α antagonist and a CD95L antagonist, (b) a soluble TNF-α receptor and a soluble CD95 receptor or portions thereof, or (c) mixtures of one or both compounds from (a) and (b).

2. The medicament according to claim 1, wherein the antibody is a monoclonal antibody or a fragment thereof.

3. The medicament according to claim 2, wherein the monoclonal antibody is an antibody derived from an animal, a humanized antibody or a chimeric antibody or a fragment thereof.

4. The medicament according to any one of claims 1 to 3, further comprising a thrombolytic agent.

5. Use of a medicament containing one or more compounds which inhibit the biological effect of TNF-α and CD95L, for preventing or treating an apoplectic stroke, a heart attack or degenerative disease, particularly Alzheimer's disease, Parkinson's disease and spinal trauma.
